(19) United States
(12) Patent Application Publication
(10) Pub. No.: US 2023/0210891 A1
(43) Pub. Date: Jul. 6, 2023

(54) POLYSACCHARIDE COMPOSITIONS AND THERAPEUTIC GELS

(71) Applicant: The University of Birmingham, Birmingham (GB)

(72) Inventors: Hannah Botfield, Birmingham (GB); Lisa Hill, Birmingham (GB); Richard Moakes, Birmingham (GB); Liam Grover, Birmingham (GB)

(21) Appl. No.: 18/009,566

(22) PCT Filed: Jun. 11, 2021
(86) PCT No.: PCT/GB2021/051458

§ 371 Date: Dec. 9, 2022

(30) Foreign Application Priority Data
Jun. 11, 2020 (GB) 2008919.9

(51) Int. Cl. A61K 31/737; A61K 31/727; A61K 9/00; A61P 27/06; A61K 47/36

(52) U.S. Cl. CPC A61K 31/737; A61K 9/0048; A61K 31/727; A61K 47/36; A61P 27/06

(57) ABSTRACT

Provided are pharmaceutical compositions comprising a sulphated polysaccharide and a shear-thinning fluid gel. The sulphated polysaccharide may, by way of example, be selected from the group consisting of: a dextran sulphate having an average molecular weight of 10,000 Da or less; heparan sulphate; fucoidan; poligeenan; furcellaran; and a carrageenan. The shear-thinning fluid gel may comprise a microgel particle forming polymer, suitably selected from one or more of the following groups: gellans; alginates; carrageenans; agarose; chitosan; pectin; agarose; agar or gelatin. The compositions disclosed may be used for the prevention and/or treatment of glaucoma. These compositions may be used for the inhibition or reduction of fibrosis. The invention also relates to the medical uses of sulphated polysaccharides, and the medical uses of shear-thinning fluid gels.

### FIELD OF THE INVENTION

The present invention relates to pharmaceutical compositions. These compositions may be used for the prevention and/or treatment of glaucoma. These compositions may be used for the inhibition or reduction of fibrosis. The invention also relates to the medical uses of sulphated polysaccharides, and the medical uses of shear-thinning fluid gels.

### INTRODUCTION

Glaucoma is a group of degenerative diseases of the eye that lead to irreversible blindness because of permanent damage to the retina. The main risk factor for developing glaucoma is raised intraocular pressure, which itself is a result of diminished drainage from the eye caused by fibrosis of the Schlemm’s canal and/or the trabecular meshwork. The drugs used to treat this condition do so by modifying production of fluids in the eye and do not reverse the underlying cause of the disease - the fibrosis.

### BRIEF DESCRIPTION OF THE DRAWINGS

FIG. 1 is a graph showing flow profiles for gellan sols with varying NaCl concentrations undergoing sheared gelation. Plateaus can be seen at higher and lower temperatures. Plateaus at higher temperatures are systems in their sol phase, plateaus at lower temperatures show equilibrium between shear and particle formation and exponential growth in between highlights the gelation profile (formation of particles).

FIG. 2 is a phase contrast micrograph of gellan fluid gel diluted in PEG. The scale bar represents 100 µm.

FIG. 3 is a graph showing the flow profile for a gellan fluid gel eye drop, showing time dependent data for increasing and decreasing shear rates.

FIG. 4 is a graph showing mechanical data for the gellan based fluid gel eye drop as a function of time after large deformational shear. The left axis shows the recovery of an elastically dominated structure (G′>G″), while the right axis shows the change in materials phase angle (45 ° = the gelling point).

FIG. 5 is a photograph of a gellan fluid gel eye drop being dispensed from a single use applicator (the fluid gel is dyed for visualisation purposes).

FIG. 6 shows the material storage modulus (left panel) and the viscosity (right panel), of commercially available eye drops. Dotted lines show values for gellan based eye drops as a comparator.

FIG. 7 sets out OCT images of an eye before (left) and after (right) the application of a fluid gel eye drop.

FIG. 8 is a graph showing gellan fluid gel layer height on the ocular surface over a period of two hours post-application. Data set out are obtained from 3 areas of the eye (nasally, centrally and temporally).

FIG. 9 OCT images of a fluid gel eye drop (left hand panels) compared to a low viscosity fluid (PBS - right hand panels) over 90 minutes. Images show that the gellan is retained for the entire time frame, whereas the PBS is removed.

FIG. 10 sets out cumulative release profiles for dextran (left hand panel) and a chemically modified dextran (blue - right hand panel) from various fluid gel matrices.

FIG. 11 is a graph showing collagen fibrillogenesis data for varying concentrations of a sulphated dextran (heparan sulphate). The line linking data points is to guide the eye and dotted black line highlights the EC₅₀ concentration.

FIG. 12 is a graph showing change in inter-ocular pressure in an animal model of glaucoma provided with a modified dextran sulphate (ILB) in a non-fluid gel topical formulation.

FIG. 13 is a graph showing change in inter-ocular pressure in an animal model of glaucoma provided with a modified dextran sulphate (ILB) in an injectable formulation.

FIG. 14 is a graph showing change in inter-ocular pressure in an animal model of glaucoma provided with a modified dextran sulphate (ILB) in a topical fluid gel formulation (an experimental version of a composition of the invention).

FIG. 15 is a graph showing change in inter-ocular pressure in an animal model of glaucoma provided with a gellan fluid gel eye drop on its own (i.e. without the modified dextran sulphate present when generating the results shown in FIG. 14).

FIG. 16 is a graph showing that the sulphated polysaccharides carrageenan and heparan sulphate are able to inhibit collagen fibrillogenesis (indicative of an anti-fibrotic activity) in a dose-dependent manner.

FIG. 17 is a graph comparing the intraocular pressure in an animal model of glaucoma treated with eyedrops representing the standard of care (dashed line) versus the untreated animal model (solid line).

FIG. 18 is a graph comparing the intraocular pressure in an animal model of glaucoma treated with eyedrops of an exemplary composition of the invention comprising dextran sulphate (dashed line) versus the untreated animal model (solid line).

FIG. 19 is a graph comparing the intraocular pressure in an animal model of glaucoma treated with eyedrops of an exemplary composition of the invention comprising fucoidan (dashed line) versus the untreated animal model (solid line).

FIG. 20 is a graph comparing the intraocular pressure in an animal model of glaucoma treated with eyedrops of an exemplary composition of the invention comprising heparan sulphate (dashed line) versus the untreated animal model (solid line).

FIG. 21 is a graph comparing the intraocular pressure in an animal model of glaucoma treated with either eyedrops representing the standard of care (dashed line) or with eyedrops of an exemplary composition of the invention comprising dextran sulphate (solid line).

FIG. 22 is a graph comparing the intraocular pressure in an animal model of glaucoma treated with either eyedrops representing the standard of care (dashed line) or with eyedrops of an exemplary composition of the invention comprising fucoidan (solid line).

FIG. 23 is a graph comparing the intraocular pressure in an animal model of glaucoma treated with either eyedrops representing the standard of care (dashed line) or with eyedrops of an exemplary composition of the invention comprising heparan sulphate (solid line).

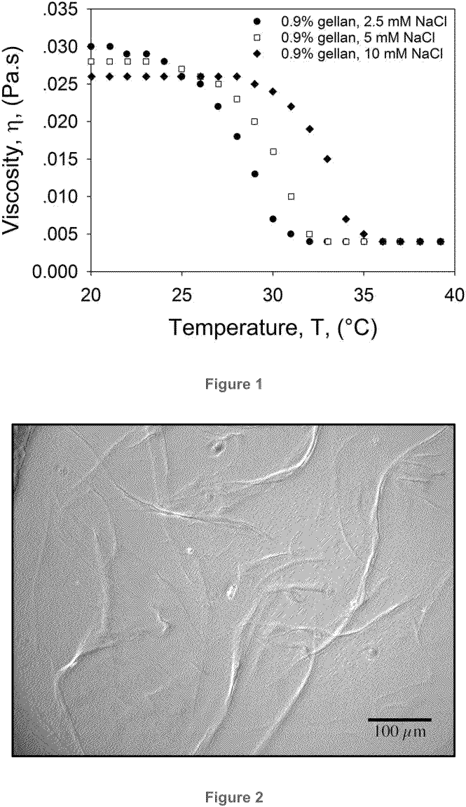

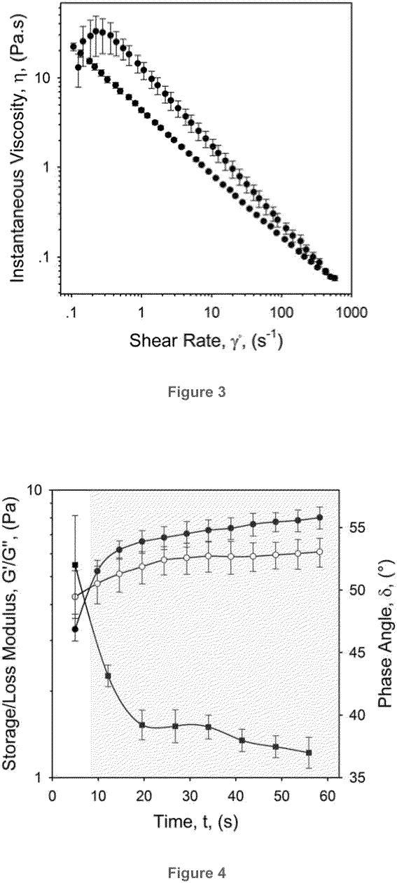

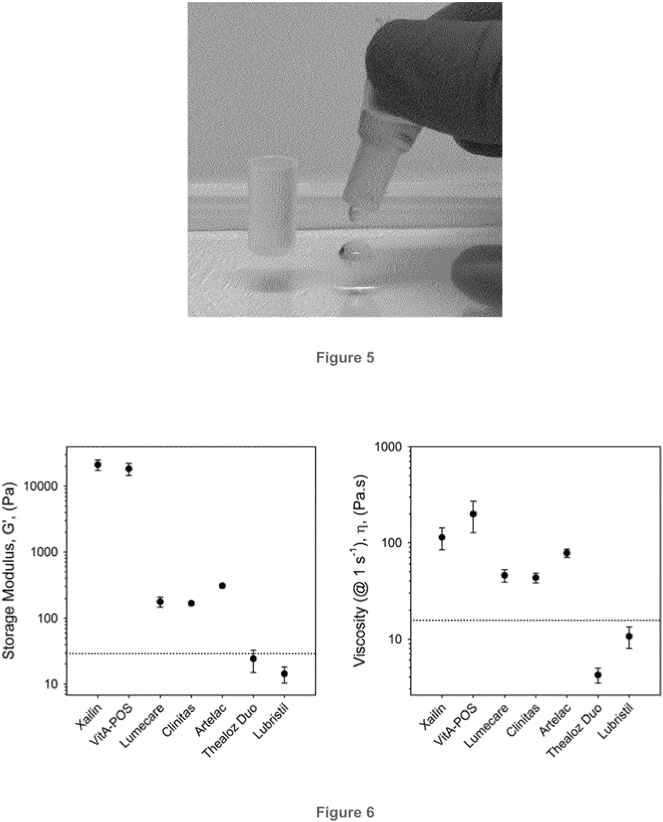

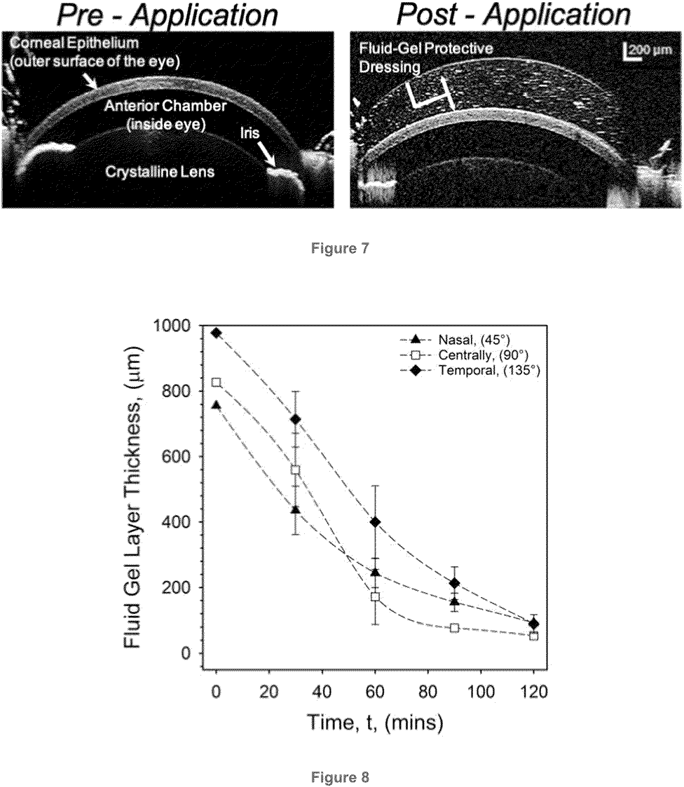

### SUMMARY OF THE INVENTION

In a first aspect the invention provides a pharmaceutical composition comprising a sulphated polysaccharide and a shear-thinning fluid gel. Compositions in accordance with the first aspect of the invention may be used for medical use in the prevention and/or treatment of glaucoma. Compositions in accordance with the first aspect of the invention may be used in methods of preventing and/or treating glaucoma in a subject requiring such prevention and/or treatment. Compositions in accordance with the first aspect of the invention may be used for medical use in the inhibition or reduction of fibrosis. Compositions in accordance with the first aspect of the invention may be used in methods of inhibiting or reducing fibrosis in a subject requiring such inhibition or reduction.

In a second aspect the invention provides a sulphated polysaccharide for use in combination with a shear-thinning fluid gel in the prevention and/or treatment of glaucoma. Suitably the shear-thinning fluid gel comprises gellan. This aspect of the invention also provides a method of preventing and/or treating glaucoma in a subject in need of such prevention and/or treatment, the method comprising providing a sulphated polysaccharide to a subject to whom a shear-thinning fluid gel has been provided.

In a third aspect the invention provides a sulphated polysaccharide for use in combination with a shear-thinning fluid gel in the inhibition or reduction of fibrosis. Suitably the shear-thinning fluid gel comprises gellan. This aspect of the invention also provides a method of inhibiting or reducing fibrosis in a subject in need of such reduction and or inhibition, the method comprising providing a sulphated polysaccharide to a subject to whom a shear-thinning fluid gel has been provided.

In a fourth aspect the invention provides a shear-thinning fluid gel for use with a sulphated polysaccharide in the prevention and/or treatment of glaucoma. Suitably the shear-thinning fluid gel comprises gellan. This aspect of the invention also provides a method of preventing and/or treating glaucoma in a subject in need of such prevention and/or treatment, the method comprising providing a shear-thinning fluid gel to a subject to whom a sulphated polysaccharide has been provided.

In a fifth aspect the invention provides a shear-thinning fluid gel for use with a sulphated polysaccharide in the inhibition or reduction of fibrosis. Suitably the shear-thinning fluid gel comprises gellan. This aspect of the invention also provides a method of inhibiting or reducing fibrosis in a subject in need of such reduction and or inhibition, the method comprising providing a shear-thinning fluid gel to a subject to whom a sulphated polysaccharide has been provided.

In a sixth aspect the invention provides a sulphated polysaccharide selected from the group consisting of: heparan sulphate; fucoidan; poligeenan; furcellaran; and a carrageenan, for use in the inhibition or reduction of fibrosis. Suitably the sulphated polysaccharide is selected from the group consisting of: heparan sulphate; and a carrageenan. Suitably the sulphated polysaccharide is a carrageenan. Suitable carrageenans are disclosed elsewhere in the specification. This aspect of the invention also provides a method of inhibiting or reducing fibrosis in a subject in need thereof, the method comprising providing the subject with a sulphated polysaccharide selected from the group consisting of: heparan sulphate; fucoidan; poligeenan; furcellaran; and a carrageenan.

In a seventh aspect the invention provides a gellan shear-thinning fluid gel for use as an eyedrop in the prevention and/or treatment of glaucoma. The eyedrop may comprise no active therapeutic agent other than the gellan shear-thinning fluid gel. This aspect of the invention also provides a method of preventing and/or treating glaucoma in a subject in need of such prevention and/or treatment, the method comprising providing to the subject a therapeutically effective amount of a gellan shear-thinning fluid gel eyedrop.

In an eighth aspect the invention provides a gellan shear-thinning fluid gel for use in the inhibition or reduction of fibrosis. The fluid gel may comprise no active therapeutic agent other than the gellan shear-thinning fluid gel. This aspect of the invention also provides a method of inhibiting or reducing fibrosis in a subject in need of such inhibition or reduction, the method comprising providing to the subject a therapeutically effective amount of a gellan shear-thinning fluid gel.

The present invention is based, to a great extent, upon the inventors’ findings that sulphated polysaccharides, particularly when used in combination with or as compositions with shear-thinning fluid gels, are notably capable of ameliorating glaucoma and fibrosis. The invention is also, in certain aspects and embodiments, based on the inventors’ findings that gellan shear-thinning hydrogels are able to ameliorate glaucoma and fibrosis even in the absence of other active therapeutic agents.

The compositions and medical uses disclosed herein are able to target the fibrosis that causes glaucoma, and so have the potential to reverse fibrosis within the drainage structures of the eye. This means that they provide the prospect of treatment that will provide a cure rather than relieving symptoms only. Current treatments for this kind of glaucoma must be maintained for the lifetime of the patient. A curative therapy of the sort described herein would mean that patients need treatment only until fibrosis is reversed.

The invention will now be further explained with reference to the following paragraphs that define certain terms used herein.

### DETAILED DESCRIPTION OF THE INVENTION

### A Sulphated Polysaccharide

Sulphated polysaccharides are employed in various aspects of the invention, whether in the compositions of the invention, or the medical uses or methods of treatment disclosed herein.

A suitable sulphated polysaccharide may have an average sulphate number per repeating saccharide unit of between 1 and 3.

In a suitable embodiment, the sulphated polysaccharide is selected from the group consisting of: a dextran sulphate having an average molecular weight of 10,000 Da or less; heparan sulphate; fucoidan; poligeenan; furcellaran; and a carrageenan.

In the case of heparan sulphate the average sulphate number per disaccharide unit may be between 2.4 and 2.7.

In the case of fucoidan the repeating saccharide unit is a fucose unit. The average sulphate number per fucose unit may be between 1 and 2. Suitably the average sulphate number pre fucose unit may be 1 or 2.

In the case of carrageenan or heparan sulphate the repeating saccharide unit may be a disaccharide unit. In the case of kappa carrageenan the average sulphate number per disaccharide unit is 1. In the case of iota carrageenan the average sulphate number per disaccharide unit is 2. In the case of lambda carrageenan the average sulphate number per disaccharide unit is 3.

Further details of dextran sulphates and alternative sulphated polysaccharides that may be of use in the various aspects of the invention are set out below.

### A Dextran Sulphate

Except for where the context requires otherwise, for example in the case of a specific alternative named polysaccharide, any reference in the present specification to a sulphated polysaccharide may be taken as a reference to a dextran sulphate having an average molecular weight of 10,000 Da (10 kDa) or less.

A suitable dextran sulphate, which may be a dextran sulphate as defined in any of the other paragraphs of this section, may have a number average molecular weight, Mn, as measured by nuclear magnetic resonance spectroscopy with an interval of 1850 and 3,500 Da. Suitably the dextran sulphate has a number average molecular weight, Mn, as measured by nuclear magnetic resonance spectroscopy with an interval of 1850 and 2,500 Da. Suitably the dextran sulphate has a number average molecular weight, Mn, as measured by nuclear magnetic resonance spectroscopy with an interval of 1850 and 2,300 Da.

A suitable dextran sulphate, which may be a dextran sulphate as defined in any of the other paragraphs of this section, may have an average sulphate number per glucose unit of between 2.5 and 3.0. Suitably the dextran sulphate has an average sulphate number per glucose unit of between 2.5 and 2.8. Suitably the dextran sulphate has an average sulphate number per glucose unit of between 2.6 and 2.7.

A suitable dextran sulphate, which may be a dextran sulphate as defined in any of the other paragraphs of this section, may have an average sulphation of C2 position in the glucose units of the dextran sulphate of at least 90%. Suitably the dextran sulphate may have an average sulphation of C2 position in the glucose units of the dextran sulphate of at least 95%.

A suitable dextran sulphate, which may be a dextran sulphate as defined in any of the other paragraphs of this section, may have an average sulphate number at the C2, C3 and C4 position in the glucose units of the dextran sulphate within an interval of 2.2 and 2.6. Suitably the dextran sulphate has an average sulphate number at the C2, C3 and C4 position in the glucose units of the dextran sulphate within an interval of 2.3 and 2.5.

A suitable dextran sulphate, which may be a dextran sulphate as defined in any of the other paragraphs of this section, may have an average number of glucose units within an interval of 4.0 and 6.0. Suitably the dextran sulphate has an average number of glucose units within an interval of 4.5 and 5.5. Suitably the dextran sulphate has an average number of glucose units within an interval of 5.0 and 5.2.

A suitable dextran sulphate, which may be a dextran sulphate as defined in any of the other paragraphs of this section, may have an average branching of glucose units that is less than 3.0%. Suitably the dextran sulphate has an average branching of glucose units that is less than 1.5%.

Except for where the context requires otherwise, references in the present disclosure to “a dextran sulphate” should also be taken as encompassing a pharmaceutically acceptable salt of such a dextran sulphate.

### Alternative Sulphated Polysaccharides

Except for where the context requires otherwise, for example in the case of a specific reference to a dextran sulphate, any reference in the present specification to a sulphated polysaccharide may be taken as a reference to any sulphated polysaccharide capable of achieving the requisite biological and therapeutic utility. For example, such a reference may be taken as referring to any of the sulphated polysaccharides described in the following paragraphs.

A suitable sulphated polysaccharide may be selected with reference to its molecular weight. In a suitable embodiment, the sulphated polysaccharide has a molecular weight of between 5 and 1,000 kDa. In suitable embodiment the sulphated polysaccharide has a molecular weight of between 8 and 800 kDa.

In a suitable embodiment the sulphated polysaccharide is selected from the group consisting of: heparan sulphate; fucoidan; poligeenan; furcellaran; and a carrageenan. A carrageenan may be further selected from the group consisting of: iota carrageenan; kappa carrageenan; and lambda carrageenan.

It will be appreciated that the molecular weights of these alternative sulphated polysaccharides vary widely. Heparan sulphate may have a molecular weight of approximately 8 kDa. Fucoidan may have a molecular weight of approximately 20 kDa. Poligeenan may have a molecular weight of between 20 and 30 kDa. Furcellaran may have a molecular weight of between 20 and 80 kDa. A suitable carrageenan may have a molecular weight of between 100 and 800 kDa.

A suitable sulphated polysaccharide may be selected with reference to its estersulphate content. A suitable sulphated polysaccharide may comprise between 15% and 40% estersulphate by weight. A suitable sulphated polysaccharide may comprise between 20% and 35% estersulphate by weight. In a suitable embodiment the sulphated polysaccharide may be selected from the group consisting of: poligeenan; furcellaran; and a carrageenan.

A suitable carrageenan may be selected from the group consisting of: iota carrageenan; kappa carrageenan; and lambda carrageenan.

Merely by way of illustration kappa carrageenan comprises approximate 20% estersulphate by weight, while iota carrageenan comprises approximate 32% estersulphate by weight, and lambda carrageenan comprises approximate 35% estersulphate by weight.

The inventors, for the first time, disclose herein that a sulphated polysaccharide selected from the group consisting of: heparan sulphate; and a carrageenan may be used in the inhibition or reduction of fibrosis. As shown demonstrated in the Examples, these sulphated polysaccharides are able to inhibit collagen fibrillogenesis in a dose dependent manner. Deposition of extracellular matrix components, such as collagen, and associated processes, such as fibrillogenesis, are essential to the initiation and development of fibrosis. Accordingly, this finding that carrageenans (such as kappa carrageenan and/or iota carrageenan) and heparan sulphate are able to disrupt collagen fibrillogenesis indicates their suitability for medical use to inhibit or reduce fibrosis.

A further aspect of the invention provides a carrageenan for use in the inhibition or reduction of fibrosis. The carrageenan may be kappa carrageenan or iota carrageenan. The carrageenan may be topically administered.

A still further aspect of the invention provides heparan sulphate for use in the inhibition or reduction of fibrosis. The heparan sulphate may be topically administered.

### A Fluid Gels and Microgels

The term “fluid gel” is used herein to refer to a suspension of microgel particles dispersed within an aqueous medium, which interact to give solid-like properties at rest, but reversibly flow under large deformation (e.g. mechanical shear).

The term “microgel” is used herein to refer to a microscopic particle of gel formed from a network of microscopic filaments of polymer.

Further details of the properties of suitable shear-thinning fluid gels are set out elsewhere in the present specification. Amongst the various the shear-thinning fluid gels disclosed herein, such a fluid gel comprising approximately 0.9% w/v of gellan may be of particularly utility in any appropriate aspect of the invention, the inventors having found that such fluid gels have unexpected benefits in the prevention and/or treatment of fibrosis, and in the inhibition or reduction of fibrosis.

### Prevention and/or Treatment of Glaucoma

In a suitable embodiment, a composition of the invention is for use in the prevention and/or treatment of glaucoma. Other aspects of the invention relate to medical uses or methods of treatment for the prevention and/or treatment of glaucoma.

References in the present disclosure to the prevention of glaucoma should be taken as encompassing prophylactic therapy. It will be appreciated that such prevention may be employed before detectable symptoms of glaucoma arise. A subject requiring prevention of glaucoma may be identified as such on the basis of genetic predisposition to glaucoma, or on the basis of other factors that give rise to an elevated risk of the development of glaucoma.

References in the present disclosure to the treatment of glaucoma should be taken as encompassing any therapy that is effective to alleviate existing glaucoma. Treatment may give rise to partial or complete relief of one or more symptoms of glaucoma. A subject may be identified as likely to benefit from treatment of glaucoma on the basis of the presence of one or more such symptom.

As demonstrated in the Examples section, exemplary compositions of the invention are able to significantly reduce intraocular pressure in an experimental model of glaucoma, illustrating their suitability for use in the prevention and/or treatment of this condition.

Furthermore, and as discussed in more detail below, the compositions, medical uses, or methods of treatment of the invention are also able to beneficially influence the pathological processes underlying glaucoma (in particular those driven by fibrosis). This enables curative treatment of glaucoma in a manner that cannot currently be achieved by therapies known to the art.

### Inhibition or Reduction of Fibrosis

In a suitable embodiment, a composition of the invention is for use in the inhibition or reduction of fibrosis. Other aspects of the invention relate to medical uses or methods of treatment for the inhibition or reduction of fibrosis.

For the purposes of the present disclosure inhibition of fibrosis should be taken as encompassing the prevention of initiation or slowing of progression of fibrosis (such that the degree of fibrosis occurring after treatment is inhibited as compared to the degree of fibrosis that would occur if not treatment is provided).

For the purposes of the present disclosure reduction of fibrosis should be taken as encompassing reversal or resolution of fibrosis that has already occurred (such that the level of fibrosis after treatment is reduced as compared to that which was present prior to treatment).

Aspects or embodiments of the invention relating to the inhibition of reduction of fibrosis have use in a wide range of therapeutic applications, including, but not limited to, the prevention and/or treatment of glaucoma (as considered in more detail elsewhere in the specification). Suitably the fibrosis to be inhibited or reduced is fibrosis associated with glaucoma. Suitably the fibrosis to be inhibited or reduced is associated with scarring. In such an embodiment the scarring may be as a consequence of healing of surgical or non-surgical wounds. Suitably the fibrosis to be prevented or treated is associated with a fibrotic disorder.

It is recognised that fibrosis results in deleterious effects in many clinical contexts. For example, fibrosis of the eye may be associated with loss of sight, and risk of blindness, while fibrosis in the skin, for example as a result of scarring, may be associated with reduced mobility, discomfort, and disfigurement (which may give rise to psychological difficulties).

Fibrosis may also give rise to complications, and hence reduced effectiveness, in surgical procedures. Merely by way of example, fibrosis that occurs after surgical insertion of stents (such as for the treatment of glaucoma) may fully or partially occlude the passageway in the stent, thus rendering the surgery ineffective.

It will be appreciated that “inhibition or reduction of fibrosis” encompasses both partial inhibition or reduction of fibrosis and complete inhibition or reduction of fibrosis. Suitable values relating to the extent to which fibrosis may be inhibited in accordance with the invention are described further below.

Compositions or medical uses of the invention may be useful in the inhibition or reduction of fibrosis or scarring at many body sites. Merely by way of example, the compositions or medical uses of the invention may be used in the inhibition or reduction of: fibrosis in the eye; fibrosis in the skin; fibrosis in the muscles or tendons; fibrosis in the nerves; fibrosis of internal organs, such as the liver or lungs; or the formation of adhesions, such as surgical adhesions or omental adhesions.

Fibrosis in the eye, of the sort that may be inhibited by the medical use of compositions or medical uses of the invention, includes fibrosis of the cornea, fibrosis of the retina, fibrosis of the ocular surface, and fibrosis in and around the optic nerve. Whilst the compositions or medical uses of the invention are suitable for topical use, it will be appreciated that agents administered topically may have an effect on the internal anatomy. Thus, compositions administered to the surface of the eye may be effective in inhibiting intraocular fibrosis.

Fibrosis in the eye that may be inhibited by the medical use of compositions of the invention may also include fibrosis associated with infection or with glaucoma.

Keratitis may also arise as a result of injury, or of disorders including autoimmune diseases such as rheumatoid arthritis or Sjogren’s syndrome. The compositions and methods of the invention may also be used in inhibiting fibrosis occurring as a result of these causes.

Fibrosis in the eye that may be inhibited by the medical use of compositions of the invention may also include fibrosis associated with surgery, such as surgery for the insertion of stents (for example in the treatment of glaucoma); and surgical procedures such as LASIK or LASEK surgery, and fibrosis associated with accidental injuries.

The skilled person will be aware of many suitable methodologies that allow the identification and quantification of fibrosis. These methodologies may also be used to identify inhibition or reduction of fibrosis. Thus they may be used to illustrate the effective medical use of the compositions of the invention, to identify therapeutically effective doses in the medical uses or methods of treatment disclosed herein.

The skilled person will be aware that there are many parameters by which the inhibition or reduction of fibrosis in the eye can be assessed. Some of these, such as induction of myofibroblast or ECM components, are also common to body sites outside the eye, while others are specific to the eye.

Fibrosis is also associated with the expression and deposition of ECM constituents. The amount of ECM deposited may be increased in fibrosis, and the arrangement of the ECM may be different from that found in undamaged comparator tissue. The data presented in the Examples illustrate that treatment using compositions of the invention gives rise to tissues in which the arrangement of ECM components more closely resembles that of unwounded tissue, thus illustrating the utility of these compositions in the inhibition or reduction of fibrosis.

Compositions or medical uses of the invention may be used in the inhibition or reduction of fibrosis associated with dermal wounds. A suitable dermal wound may be selected from the group consisting of: a burn; an incision; an excision; an abrasion; a chronic wound; and a wound arising from the body’s reaction to a stimulus. Examples of this latter category include systemic chemical and/or allergic reactions that cause skin to blister severely and to shed, as well as genetic-related diseases that result in compromised skin structure and homeostasis. These reactions or diseases may lead to skin blistering, peeling and dramatically increased risk and severity of wounding (even from relatively minor contact). Examples of such diseases include epidermolysis bullosa (for example epidermolysis bullosa simplex, junctional epidermolysis bullosa, or dystrophic epidermolysis bullosa) and Kindler syndrome. The compositions or methods of the invention are suitable for use in inhibition or reduction of fibrosis in subjects having such diseases.

The compositions of the invention are suitable for use at sites of surgical incisions, to inhibit fibrosis that may otherwise be associated with the healing of such surgical wounds.

A composition or medical use of the invention may be able to achieve an inhibition or reduction of fibrosis of at least 5% as compared to a suitable control agent. For example, a composition or medical use of the invention may be able to achieve an inhibition or reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a suitable control agent. A composition of the invention may be able to achieve substantially total inhibition or reduction of fibrosis as compared to a suitable control agent.

By the same token, the medical use of compositions of the invention, or medical uses or methods of treatment using such compositions, to inhibit or reduce fibrosis may achieve an inhibition or reduction of at least 5% as compared to a suitable control. For example, such medical uses or methods of treatment may achieve an inhibition or reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a suitable control. The medical uses or methods of treatment of the invention may achieve substantially total inhibition or reduction of fibrosis as compared to a suitable control.

The selection of a suitable control will be readily determined by one of skill in the art. Merely by way of example, a suitable control for assessment of the ability of a composition of the invention to inhibit fibrosis in the eye may be provided by the recognised standard of care, or an experimental proxy thereof.

### Topical Medicament

In a suitable embodiment, a composition of the invention is for use as a topical medicament. Similarly, the medical uses of the invention may involve provision of one or more of the recited therapeutic agents in a topical medicament. Such a topical medicament may be used in the treatment of glaucoma. Such a topical medicament may be used in the inhibition or reduction of fibrosis.

While examples of sulphated polysaccharides have been known for the treatment of glaucoma, these prior therapeutic uses have involved injection of the sulphated polysaccharide, for example via subcutaneous or intravenous injection. As demonstrated in the Examples, dextran sulphates are not effective in the treatment of glaucoma when administered topically by traditional routes, for example in the form of non-fluid gel eyedrops. Surprisingly, however, the inventors have found that the therapeutic effectiveness of sulphated polysaccharides is significantly improved if they are incorporated in a pharmaceutical composition comprising a shear-thinning fluid gel. These novel compositions markedly increase the ability of the topically administered sulphated polysaccharides to reduce intraocular pressure, thereby rendering the sulphated polysaccharides suitable for use as therapeutic agents in the prevention and/or treatment of glaucoma.

It will be appreciated that topical treatments, such as eyedrops, are much easier and more comfortable for a patient to self-administer than are injections, whether subcutaneous or intravenous. Even when administered by another, such as a physician, there is less pain associated with the use of topical treatments. Improvements in ease and comfort of this sort not only beneficially impact the patient’s experience and quality of life, but also increase compliance with treatment regimes, and thus improve therapeutic effectiveness.

For the avoidance of doubt, it will be appreciated that previously disclosed compositions for treatment of glaucoma, in which a sulphated polysaccharide is an active agent, have not made use of a sulphated polysaccharide in composition with or in combination with a shear-thinning fluid gel. Shear-thinning fluid gels of the sort used in the composition of the present invention are not typically employed in injectable compositions. Indeed, the properties of shear-thinning fluid gels that make them suitable for use in the context of the present invention (as topical medicaments such as eyedrops) may not be desirable in injectable medicaments, where the composition (once injected and no longer subject to shear) will take on a more solid consistency.

### Topical Eyedrop

In a suitable embodiment, a composition of the invention for use as a topical medicament in the treatment of glaucoma is formulated as an eyedrop.

As noted above, the inventors have surprisingly found that compositions of the invention, comprising a sulphated polysaccharide and a shear-thinning fluid gel, provide therapeutic effectiveness that is not conferred by other eyedrop formulations.

Similarly, shear-thinning fluid gels comprising gellan (for example at a concentration of approximately 0.9% w/v) demonstrate unexpected efficacy in experimental models of glaucoma.

The shear-thinning fluid gels of the compositions or medical uses of the invention are particularly well suited to use as eyedrops. In particular, shear-thinning fluid gels can be produced such that the gel is liquid when under application of shear (such as when being forced through an eyedropper) but then takes on a more solid consistency (for example similar to that of a soft contact lens) when shear is removed. Thus the composition is liquid when dispensed from an eyedropper, and so able to spread over the corneal surface of an eye to which it is applied. Once the composition has spread it takes on a more solid consistency, which retains the composition in situ. In a suitable embodiment such a composition is able to act as a depot for release of the sulphated polysaccharide.

In particularly advantageous formulation, an eyedrop composition of the invention may be formulated such that the surface of the composition in situ is rendered liquid by the sweep of the eyelid during blinking. In embodiments of the compositions of the invention, such formulations enable pulsatile delivery of the sulphated polysaccharide over time, which facilitates the maintenance of a therapeutically effective amount of the sulphated polysaccharide at the site requiring treatment.

As set out elsewhere shear-thinning fluid gels comprising gellan are of particularly utility as topical eyedrops in connection with the compositions, medical uses, or methods of treatment of the invention. Such applications may be without other therapeutically active agents, or in combination or in composition with sulphated polysaccharides, as discussed elsewhere.

### Prevention and/or Treatment of Glaucoma by Inhibition or Reduction of Fibrosis

In a suitable embodiment, a composition of the invention or medical use of the invention is for use in the prevention and/or treatment of glaucoma by inhibition or reduction of fibrosis.

As mentioned in the introduction, fibrosis underlies the development of glaucoma, particularly fibrosis of Schlemm’s canal and/or the trabecular meshwork. Current glaucoma therapies do not seek to address this fibrosis, but instead treat the condition by modifying production of fluids in the eye (with a view to reducing symptoms of glaucoma, such as intraocular pressure).

Although sulphated polysaccharides have previously been proposed as active agents for use in the treatment of glaucoma, until now it has been believed that they achieved their effect by addressing the symptoms of glaucoma. In contrast to this, the inventors have found that compositions of the invention comprising sulphated polysaccharides and shear-thinning fluid gels, or gellan shear-thinning fluid gels, are able to treat glaucoma by inhibiting and/or reducing fibrosis associated with the development and progression of this disease.

In view of the above, it will be appreciated that the use of the sulphated polysaccharides as active agents for the treatment of glaucoma by inhibition or reduction of fibrosis constitutes a mode of action for these therapeutic agents that has not previously been suggested. Similarly, this mode of action has not been described before in connection with gellan shear-thinning fluid gel eyedrops.

The skilled person will recognise that the finding that compositions of the invention (for example those comprising sulphated polysaccharides in shear-thinning fluid gels, or gellan shear-thinning fluid gels) are able to treat glaucoma by inhibiting or reducing fibrosis opens new clinical applications, since it identifies the compositions of the invention as agents able to target the underlying pathology of glaucoma, rather than simply able to reduce symptoms associated with this disease, such as intraocular pressure.

It will be recognised that this finding enables use in new therapeutic contexts. For example the composition or medical uses of the invention may be employed in the curative treatment of glaucoma, rather than alleviation of symptoms associated with glaucoma.

### Shear-Thinning Fluid Gels

The term “shear-thinning” is used herein to define the fluid gel compositions of the present invention. This terminology is well understood in the art and refers to fluid gel compositions that have a viscosity that reduces when a shear force is applied to the fluid gel. The shear-thinning fluid gel compositions of the invention possess a “resting” viscosity (in the absence of any applied shear force), and a lower viscosity when a shear force is applied. This property of fluid gel compositions enables them to flow and be administered to the body when a shear force is applied (for example, by applying a force to a tube or dispenser containing the fluid gel composition of the invention). Once applied under the application of shear, and the applied shear force is removed, the viscosity of the fluid gel composition increases. Typically, the fluid gel compositions of the present invention will have a viscosity of below 1 Pa.s when subjected to a shear force to administer the hydrogel composition. At viscosities below 1 Pa.s, the fluid gel composition will be capable of flowing. The resting viscosity will typically be above 1 Pa.s, for example greater than 2 Pa.s, greater than 3 Pa.s, or greater than 4 Pa.s.

In a suitable embodiment of a composition of the invention the shear-thinning fluid gel has a viscosity of 0.1 Pa.s or greater when exposed to zero shear and the viscosity reduces when the shear-thinning fluid gel is subjected to shear.

Suitably the shear-thinning fluid gel has a viscosity of 5 Pa.s or greater when exposed to zero shear and the viscosity reduces to below 1 Pa.s when the shear-thinning fluid gel is subjected to shear.

In a suitable composition the shear-thinning fluid gel comprises a shear-thinning hydrogel composition comprising 0.1 to 10% w/v (such as 0.1 to 5% w/v, or 0.1 to 2.5% w/v) of a microgel particle forming polymer dispersed in an aqueous medium, wherein the viscosity and/or elastic modulus of the shear-thinning composition reduces when the hydrogel is exposed to shear.

In a suitable composition the shear-thinning hydrogel composition further comprises 0.5 to 100 mM of a monovalent and/or polyvalent metal ion salt as a cross-linking agent.

In a suitable composition the shear-thinning fluid gel comprises a microgel particle forming polymer selected from one or more of the following groups: gellans; alginates; carrageenans; agarose; chitosan; pectin; agarose; agar or gelatin.

In a suitable composition of the invention the shear-thinning fluid gel comprises gellan. In a suitable example the shear-thinning fluid gel comprises approximately 0.9% w/v of gellan.

In a suitable composition the shear-thinning fluid gel comprises a synthetic microgel particle forming polymer selected from one or more of: polyols (such as polyalkylene glycols); polyamides; polyesters; polyalkylenes; polystyrenes and polyacrylates.

In a suitable embodiment, the shear-thinning fluid gel is non-sulphated. For example, in a suitable composition the shear-thinning fluid gel comprises a microgel forming polymer that is a non-sulphated polymer. Suitably, all (or substantially all) of the microgel particle forming polymer present in the shear-thinning fluid gel of a composition of the invention may be a non-sulphated polymer.

The inventors have found that the use of non-sulphated shear-thinning gels to deliver sulphated polysaccharides in the manner described herein provides surprising advantages in terms of improved therapeutic activity when compared to that provided by the sulphated polysaccharide on its own. Without wishing to be bound by any hypothesis, the inventors believe that this effect may arise as a result of the non-sulphated shear-thinning gels binding an increased amount of the aqueous solution comprising the therapeutically effective sulphated polysaccharide molecules, which enables higher local concentrations of these molecules to be delivered to the body surface to which the composition of the invention is provided.

### Pharmaceutical Compositions

The following provides a number of exemplary methods for formulation of pharmaceutical compositions of the invention comprising a sulphated polysaccharide and a shear-thinning fluid gel

### Methodology for the Preparation of Composition Comprising a Sulphated Polysaccharide Active Agent in a Shear-Thinning Fluid Gel Set by Temperature

These protocols have 3 different points at which the active can be added: “*”, “**” and “***”.

### For Actives Stable in PBS

a. Prepare the active (carrageenan (iota, kappa, lambda); dextran or dextran derivative; heparan sulphate) in PBS so that when mixed (20 times dilution) the final concentration is within the required dose range (0.005 - 0.5% (w/v)).

### This Range Has Been Selected as a Range Which Encompasses the EC₅₀ Values for Iota-Carrageenan, Kappa-Carrageenan and Heparan Sulphate

b. To a mixing vessel add 90 ml of deionised water.

c. Add 5 ml of aqueous metal salt (mono or polyvalent cation, e.g. NaCl or CaCl₂) so that the final concentration in 100 ml is between 10 to 200 mM(0.2 M).

d. To the aqueous solution, add a thermally gelling biopolymer (e.g. gellan (low acyl), carrageenan (kappa), agarose, agar) so the final polymer concentration in 100 ml is between 0.5 and 2% (w/v).

e. Stir the system allowing the polymer to disperse.

f. Heat the polymer solution, to ca. 90° C., until all the polymer has dissolved.
* In the case of thermally stable actives - add 5 ml of active in PBS and allow to mix.

g. Maintain constant mixing/shearing (100-1500 rpm; 200-1000 s⁻¹), whilst allowing the mixture to cool (at a cooling rate between 0.5 to 5° C.min⁻¹) to 40° C.
** In the case of semi-thermally unstable actives - add 5 ml of active in water with continued mixing/shearing.

h. Continue cooling the mixture whilst maintaining the mixing/shear, throughout the gelation profile (to a temperature ranging between 10 and 20° C.).
*** In the case of thermally unstable actives - add 5 ml of active in water with continued mixing/shearing.

i. Stop mixing, and store.

### For Actives Unstable in PBS

a. Prepare the active in deionised water so that when mixed (20 times dilution) the final concentration is within the required dose range (0.005 - 0.5% (w/v)).

b. To a mixing vessel add 85 ml of deionised water.

c. Add 5 ml of PBS.

d. Add 5 ml of aqueous metal salt (mono or polyvalent cation, e.g. NaCl or CaCl₂) so that the final concentration in 100 ml is between 10 to 200 mM(0.2 M).

e. To the aqueous solution, add a thermally gelling biopolymer (e.g. gellan (low acyl), carrageenan (kappa), agarose, agar) so the final polymer concentration in 100 ml is between 0.5 and 2% (w/v).

f. Stir the system allowing the polymer to disperse.

g. Heat the polymer solution, to ca. 70° C., until all the polymer has dissolved.
* In the case of thermally stable actives - add 5 ml of active in water and allow to mix.

h. Maintain constant mixing/shearing (100-1500 rpm; 200-1000 s⁻¹), whilst allowing the mixture to cool (at a cooling rate between 0.5 to 5° C.min⁻¹) to 40° C.
** In the case of semi-thermally unstable actives - add 5 ml of active in water with continued mixing/shearing.

i. Continue cooling the mixture whilst maintaining the mixing/shear, throughout the gelation profile (to a temperature ranging between 10 and 20° C.).
*** In the case of thermally unstable actives - add 5 ml of active in water with continued mixing/shearing.

j. Stop mixing, and store.

### Methodology for the Preparation of Composition Comprising a Sulphated Polysaccharide Active Agent in a Shear-Thinning Fluid Gel Set by Action of Radicals

### For Actives Stable in PBS

a. Prepare a polymer solution using a radical gelling polymer (e.g. PEGDA) in PBS so that the polymer concentration is in a range between 1 to 10%.

b. Add 50 ml of the polymer solution to a stirring vessel.

c. Add the active (carrageenan (iota, kappa, lambda); dextran or dextran derivative; heparan sulphate) to the polymer solution so that the final concentration is within the required dose range (0.005 - 0.5% (w/v)).

d. Add the radical forming initiator (e.g. irgacure 1173 (UV), AIBN (Heat)) so that the concentration is the range of 0.1 to 1%.

e. Apply constant mixing/shearing (100-1500 rpm; 200-1000 s⁻¹).

f. Apply the stimulus to gelation (UV light, Heat) whilst maintaining mixing/shear until gelation has finished

g. Stop mixing, and store.

### For Actives Unstable in PBS

a. Prepare a polymer solution using a radical gelling polymer (e.g. PEGDA) in deionised water so that the polymer concentration is in a range between 1 to 10%.

b. Add 50 ml of the polymer solution to a stirring vessel.

c. Add the active (carrageenan (iota, kappa, lambda); dextran or dextran derivative; heparan sulphate) to the polymer solution so that the final concentration is within the required dose range (0.005 - 0.5% (w/v)).

d. Add the radical forming initiator (e.g. irgacure 1173 (UV), AIBN (Heat)) so that the concentration is the range of 0.1 to 1 %.

e. Apply constant mixing/shearing (100-1500 rpm; 200-1000 s⁻¹).

f. Apply the stimulus to gelation (UV light, Heat) whilst maintaining mixing/shear until gelation has finished

g. Stop mixing, and store.

### Methodology for the Preparation of Composition Comprising a Sulphated Polysaccharide Active Agent in a Shear-Thinning Fluid Gel Set by Ionotropic Action

### For Actives Stable in PBS

h. Prepare the active (carrageenan (iota, kappa, lambda); dextran or dextran derivative; heparan sulphate) in PBS so that when mixed (20 times dilution) the final concentration is within the required dose range (0.005 - 0.5% (w/v)).

### This Range has Been Selected as a Range Which Encompasses the EC₅₀ Values for Iota-Carrageenan, Kappa-Carrageenan and Heparan Sulphate

i. To a mixing vessel add 90 ml of deionised water.

j. To the water, add an ionotropically gelling biopolymer (e.g. alginate, pectin) so the final polymer concentration in 100 ml is between 0.5 and 2% (w/v).

k. Stir the system allowing the polymer to disperse.

l. Add 5 ml of the active solution to the polymer solution.

m. Prepare an aqueous metal salt (polyvalent cation, e.g. CaCl₂) solution so that the final concentration when diluted by a factor of 20 is between 10 to 200 mM.

n. Maintain constant mixing/shearing (100-1500 rpm; 200-1000 s⁻¹), whilst slowly adding the metal ion solution (at a constant rate between 0.1 to 5 ml.min⁻¹), until 5 mls has been added.

o. Stop mixing, and store.

### For Actives Unstable in PBS

a. Prepare the active in deionised water so that when mixed (20 times dilution) the final concentration is within the required dose range (0.005 - 0.5% (w/v)).

b. To a mixing vessel add 90 ml of deionised water.

c. To the water, add an ionotropically gelling biopolymer (e.g. alginate, pectin) so the final polymer concentration in 100 ml is between 0.5 and 2% (w/v).

d. Stir the system allowing the polymer to disperse.

e. Add 5 ml of the active solution to the polymer solution.

f. Prepare an aqueous metal salt (polyvalent cation, e.g. CaCl₂) solution so that the final concentration when diluted by a factor of 20 is between 10 to 200 mM.

g. Maintain constant mixing/shearing (100-1500 rpm; 200-1000 s⁻¹), whilst slowly adding the metal ion solution (at a constant rate between 0.1 to 5 ml.min⁻¹), until 5 mls has been added.

h. Stop mixing, and store.

### EXAMPLES

The invention will now be further described with reference to the following non-limiting examples.

### Study 1

### Materials and Methods

### A Preparation of Gellan Fluid Gel for IOP Experiments

### Synopsis

Gellan FG was prepared for addition ILB to study the impact of the composition on IOP in an animal model.

### Equipment

Balance
Cell stirrer flack (100 ml)
Hotplate/stirrer
Digital thermometer
Pipettes (10 ml)
Pipetboy
Small weighing boats
Spatula

### Materials

Gellan (CGLA) (Kelcogel; Lot #: 5C1623A)
NaCl (Fisher Chemicals, Lot: 1665066, opened 04/11/16)
PBS

### Method

1. Preparation of sodium chloride solution (0.2 M):
a. Add cell grade water to half fill a 100 ml volumetric flask
b. Weigh out 11.68 g of NaCl using a high precision scale
c. Add salt to the volumetric flask
d. Fill the volumetric flask to the line and invert until dissolved

2. Preparation of Gellan fluid gels:
a. To the cell stirrer flask add 90 ml by mass.
b. 5 ml of 0.2 M NaCl was added to the flask.
c. 5 ml of PBS was added to the flask.
d. Gellan (0.9 g; actual mass 0.908 g) was weighed out into a small weighing boat.
e. Gellan was added to the cell flask by pinching the weighing boat and gently knocking to prevent clumping in the water. Flask was swirled periodically to disperse the powder layer.
f. Stir the gellan and allow to disperse.
g. Flask was sealed appropriately for autoclaving and autoclaved on a media setting.
h. After autoclaving (whilst still hot), the flask was removed and stirred. Stirring was continued until the gellan sol had completely cooled to ca. 20° C.
i. Once fully cooled, stirring was stopped.

3. Packaging:
a. Gellan FG was decanted into falcon tubes using a laminar flow hood.

### B Rheology: Mechanical Characterisation of Fluid Gels for IOP

### Synopsis

Mechanical characteristics of the fluid gels were tested during and after production. Additionally, a range of commercially available eye drops were tested for their mechanical properties to compare against the fluid gel.

### Equipment

Rheometer
40 mm serrated parallel plates
Cup and vane geometry

### Materials

Gellan fluid gel formulations
“Preparation of gellan fluid gel for IOP experiments”
Commercially available eye drops.

### Method

1) Flow profile of gellan during fabrication:
   a. Load cup and vane geometry into the rheometer.
   b. Set the cup to 50° C.
   c. Add gellan sol to the cup and lower vane.
   d. Run a single shear rate with temperature ramp:
      i.500/s
      ii.50 to 20° C.
      iii.1° C./min.
2) Hysteresis of the fluid gel:
   a. Load serrated parallel plate into the rheometer
   b. Set the temperature to 20° C.
   c. Load sample and lower top plate to 1 mm gap height.
   d. Run a shear rate ramp (increasing and decreasing):
      i.0.1 to 600 /s
      ii.1 min ramp time
3) Elastic modulus recovery:
   a. Load serrated parallel plate into the rheometer
   b. Set the temperature to 20° C.
   c. Load sample and lower top plate to 1 mm gap height.
   d. Run a pre-shear followed by single frequency test:
      i. Pre-shear
         1. Shear at 600 /s for 30 s
      ii.Single frequency
         1. 1 Hz
         2. 0.5% strain
         3. 60 s
4) Commercial eye drop rheology
   a. Load serrated parallel plate into the rheometer
   b. Set the temperature to 20° C.
   c. Load sample and lower top plate to 1 mm gap height.
   d. Run a pre-shear followed by single frequency test:
      i. Conditioning step
         1 Temperature equilibrium 20° C., 1 min
         2 No shear
      ii. Amplitude sweep (strain controlled)
         0. 0.1 to 100%
         1. 1 Hz frequency
         2. 20° C.
      iii. Viscosity curve
         0. 0.1 to 600 /s
         1. 20° C.

### C Collagen Fibrillogenesis Study

### Synopsis

Collagen fibrillogenesis was undertaken using collagen acquired from Corning to assess the ability of carrageenans with different degrees of sulphation, gellan gum (LA) and heparan sulphate on their ability to alter fibrillogenesis.

### Equipment

Pipettes and Tips: 20 uL, 200 uL and 1000 uL
Multi-Channel Pipette 200 ul
96 Well Plate
Plate reader (405 nm)
Spatula
Small weighing boats
100 ml beakers (with lids or covering)
Balance
Small magnetic flea
Hotplate/stirrer

### Materials

PBS - Dulbecco’s PBS pH 7.4
Carrageenans (iota and kappa) (Sigma)
(CGLA) (Kelcogel; Lot #: 5C1623A)
Heparan sulphate (Tinzaparin - mw ~ 9000)
Collagen - Rat tail - Corning 4.04 mg/ml
Deionised water

### Method

1. Preparation of stock solutions:
a. A 0.2% (w/v) solution of both carrageenans, gellan and heparan in water were prepared. (0.1 g polymer in 50 ml water)
b. Mixtures were stirred until fully dispersed/hydrated. (prevent water loss by covering the beaker)

2. Preparation of Buffers:
a. PBS diluent buffer;
   i. To 200 ml deionised water add sodium phosphate (0.35 g) (0.348 g)and sodium chloride (2.05 g) (2.061 g)
   ii. Adjust pH to 7.40 and adjust final volume to 250 ml.
   iii. Store at room temperature.
b. PBS reaction buffer;
   i. To 200 ml deionised water add sodium phosphate (2.07 g) (2.081 g) and sodium chloride (4.09 g) (4.091 g)
   ii. Adjust pH to 7.40.
   iii. Add deionised water to create a final volume of 250 ml.
   iv. Store at room temp.

3. Collagen fibrillogenesis assay:
a. Pipette 75 ml of buffer (PBS) into rows A and B for blanks.
b. Standard curve generated in a dilution plate. Add 150 ml of each preparation into C1, D1 and E1.
c. Add 150 mL of sample to rows F1, G1 and H1.
d. Add 75 ml of buffer (PBS) to the additional columns 2 to 12.
e. Using a multi-channel pipette, preform a 2-fold dilution by transferring 75 ml from column 1 to column 2. Continue serial diluting through to column 12. Discard the extra 75 ml from column 12.
f. Calculate volume of collagen needed to make 10 ml (enough for 1 plate) at a concentration of 800 mg/ml in chilled water and gently mix. (Do not vortex) Mix by pipette or inverting - keep on ice.

Target conc. (0.8 mg/ml)Collagen stock conc. in mg/ml × 10=ml of collagen required

10 ml- ?ml of collagen required= ??ml of water required
a. Add 75 ml of collagen to each row (B to H).
b. Add 150 ml of phosphate buffer to all of the wells.
c. Incubate for 2-4 hrs at room temperature and analysed using 405 nm plate reader. (correct for the plate by using 570 nm reference)

Each sample was replicated 3 times, two polymers in each plate (2 plates total). Collagen standard and pbs only were obtained on each plate.

### Results

Data shows that fibrillogenesis can be controlled trough the addition of biopolymers. A direct link to the degree of sulphonation and ability to prevent fibrillogenesis can be drawn, with higher degrees (lota > lambda > gellan) requiring lower doses to achieve a 50% reduction in turbidity.

### D Methods for FG and FG+ILB in Vivo Studies

Fluid gel production methods were as described above.

### Methods for the in Vivo Study:

Treatment groups for CiC in vivo experiments (some unrelated groups have been omitted) All rats received twice weekly injections of 3.5 µl 5 µg/ml TGFβ1 from the start of the experiment (day 0) until the end (day 28). This is the glaucoma model.

IOP measures were taken twice weekly using a rebound tonometer, between the hours of 9-11 am (to reduce circadian fluctuations in IOP) and before TGFβ injections.

On day 14 rats then received eye drops, by the use of a pipette, twice a day, every day between day 14 and day 28.

The groups were as follows.

| Group No. | Total N (no. of rats) | Treatment |
|---|---|---|
| A-FG | 5 | 5.1 µl 0.9% saline + 5.1 µl fluid gel |
| C | 5 | 10.2 µl 9.8 mg/ml ILB |
| E | 5 | 5.1 µl 19.6 mg/ml ILB+5.1 µl fluid gel |

Order of eye drop applications as per table above, the fluid gel was applied last on each occasion.

Exemplary dextran sulphate ILB: 100 µg ILB in 5.1 µl = 19.6 mg/ml ILB solution Total volume for each eye drop was 10.2 µl

### Results

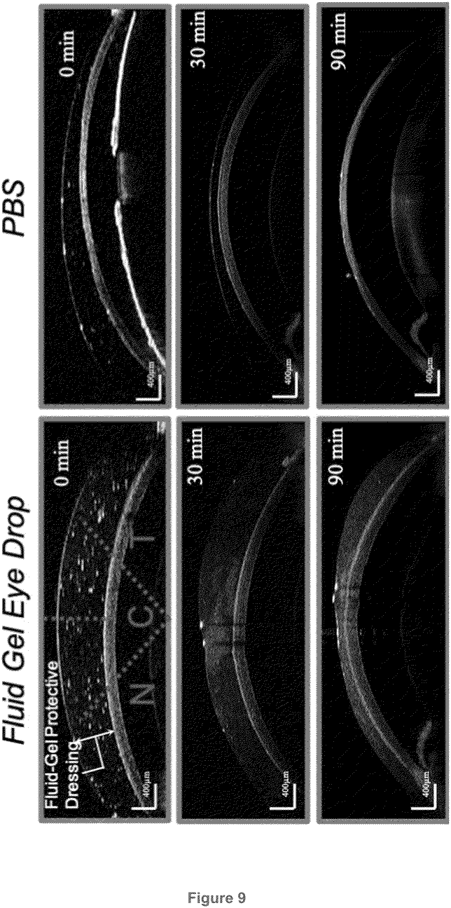

FIG. 8 demonstrates retention on the eye’s surface of eyedrops of a gellan shear-thinning fluid gel. The graph shows thickness of the eyedrop layer, at defined locations on the surface, with time.

Similar results can be seen in FIG. 9, which illustrates retention of the gellan fluid gel eyedrops for a full 90 minute monitoring period.

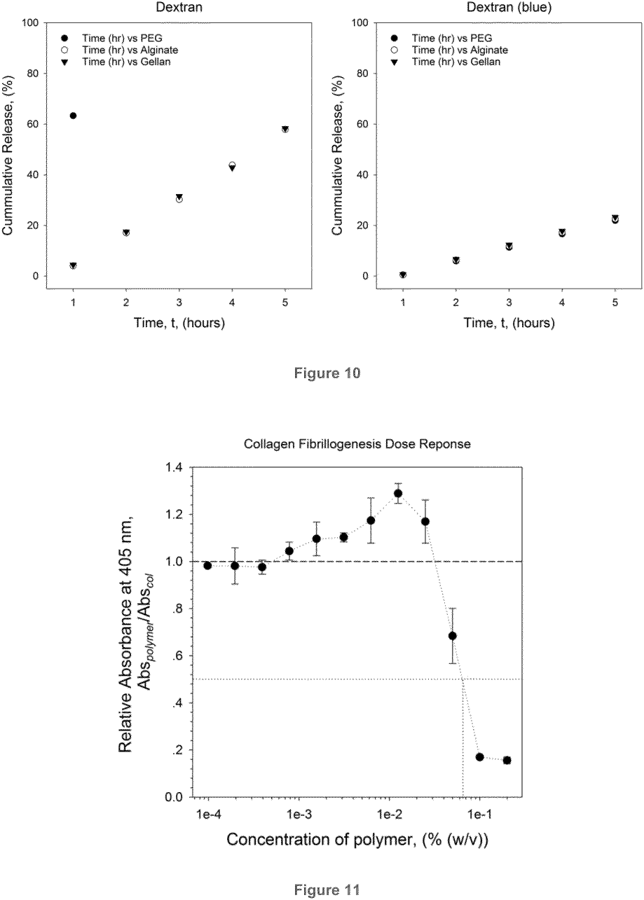

FIG. 10 illustrates cumulative release profiles for exemplary sulphated polysaccharides (dextran in left panel, and chemically modified dextran (blue) in right panel) from exemplary shear-thinning fluid gel matrices (utilising the particle forming polymers gellan, PEG and alginate). As can be seen, each of these exemplary shear-thinning fluid gels exhibits a broadly comparable release profile - indicating that a wide range of such fluid gels can be used in pharmaceutical compositions for the delivery of sulphated polysaccharides.

FIG. 11 sets out data for collagen fibrillogenesis in the presence of various concentrations of a sulphated polysaccharide (in this case a dextran sulphate). As can be seen, the sulphated polysaccharide has a dose-dependent ability to inhibit collagen fibrillogenesis, indicating utility in inhibition or reduction of fibrosis.

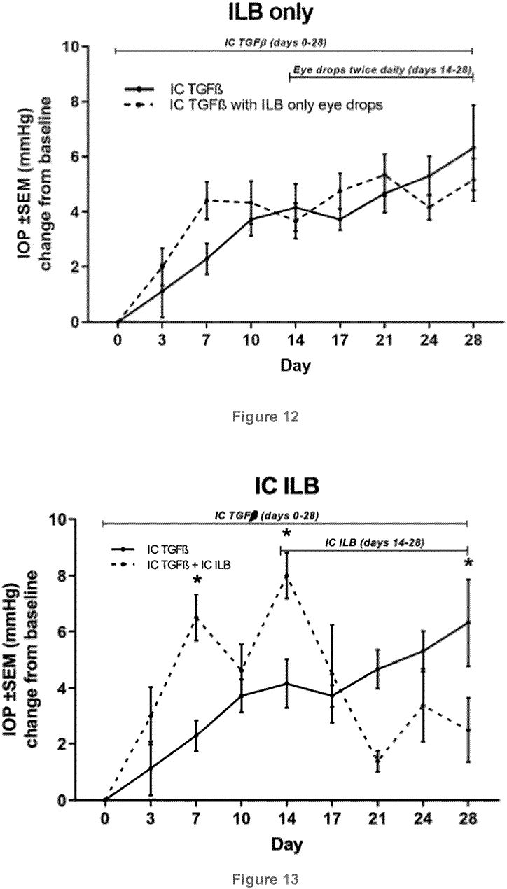

FIGS. 12 to 15 set out the results of a study investigating change in intraocular pressure (IOP) in the animal model of glaucoma described above. A dextran sulphate (designated ILB, and as described in WO 2016/076780 - the entirety of which is incorporated by reference insofar as it describes the manufacture and characterisation of this dextran sulphate) is used as an exemplary sulphated polysaccharide.

In FIG. 12, ILB was provided in a non-fluid gel eyedrop formulation. As can be seen, the dextran sulphate administered in this manner did not achieve a significant reduction in IOP as compared to controls.

In FIG. 13, ILB was provided in an injectable formulation. The results of this administration were varied, with IOP of treated animals higher than controls at certain timepoints (e.g. day 14) and lower than controls at other timepoints (e.g. day 28).

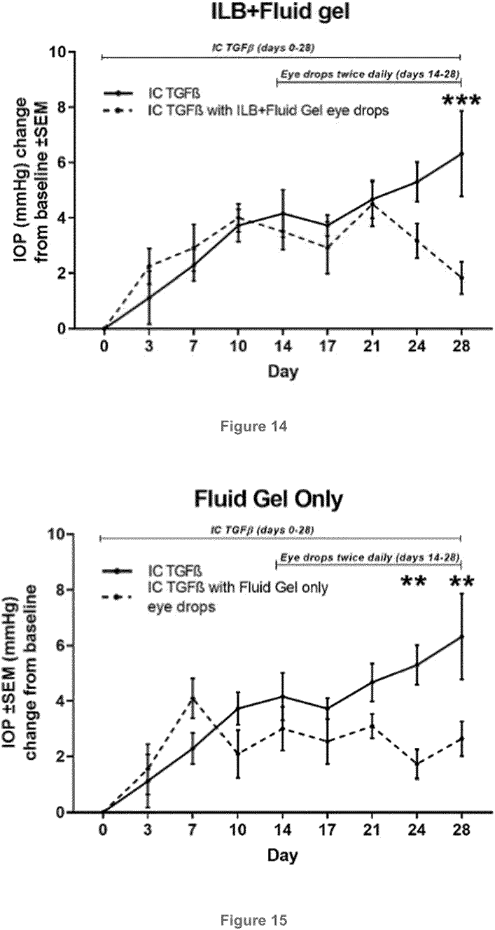

FIG. 14 shows results achieved when ILB was administered as an eyedrop, followed by administration of a shear-thinning fluid gel eyedrop afterwards (in this case a gellan fluid gel comprising approximately 0.9% w/v gellan). Here it can be seen that, with passage of time, IOP in treated animals was significantly lower than in controls. This shows that the presence of fluid gel renders the sulphated polysaccharide able to reduce IPO in a manner that is not seen in either FIG. 12 or FIG. 13, and demonstrates the suitability of this experimental treatment as a therapy for glaucoma.

FIG. 15 shows results achieved using a 0.9% w/v gellan shear-thinning fluid gel eyedrop as a therapy. Again, it can be seen that, with passage of time, IOP in treated animals was significantly lower than in controls. As above, this demonstrates the suitability of this experimental treatment as a therapy for glaucoma.

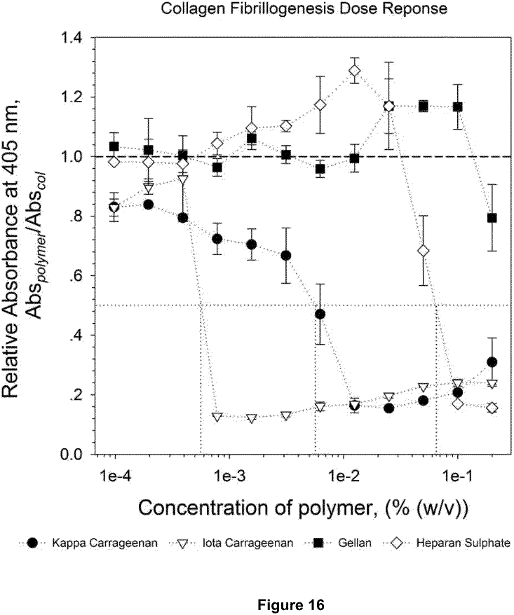

FIG. 16 shows the results of a collagen fibrillogenesis assay, comparing the effect of increasing doses of various polymers (gellan, and the sulphated polysaccharides heparan sulphate, kappa carrageenan and iota carrageenan) on fibril formation. It can be seen that the sulphated polysaccharides heparan sulphate and carrageenan both inhibit collagen fibril formation, indicating their ability to inhibit or reduce fibrosis, and thus their therapeutic utility.

### Study 2

The inventors studied the therapeutic utility of further compositions of the invention comprising sulphated polysaccharides in shear-thinning fluid gels for the treatment of glaucoma. Details of this second study are set out below.

### Experimental Design

A rat experimental model of glaucoma was used. All rats received twice weekly intracameral (IC) injections of 3.5 µl 5 µg/ml TGFβ1 from the start of the experiment (day 0) until the end (day 28). This establishes the experimental glaucoma model.

Intraocular pressure (IOP) measures were taken twice weekly using a rebound tonometer, between the hours of 9-11am (to reduce circadian fluctuations in IOP) and before TGFβ injections were administered.

On day 14 rats then also received a single 10 µl eye drop (ED) of either a composition of the invention or a composition representing the standard of care. The eyedrops were administered by pipette, twice a day, every day between day 14 and day 28. Gellan fluid gels were prepared as describe above, and the various sulphated polysaccharides investigated (dextran sulphate, fucoidan, or heparan sulphate) were mixed into the fluid gel at a concentration of 1 mg/ml prior to administration as a single formulation eye drop.

The most common eye drop prescribed clinically for the treatment of glaucoma is latanoprost 50 µg/ml (Xalatan®). Therefore, this treatment was used in this study for the standard of care (SOC).

The groups investigated in the study were as follows:

| Total N (no. of eyes) | Treatment |
|---|---|
| 6 | IC TGFβ only (model of untreated glaucoma) |
| 5 | IC TGFβ + Latanoprost ED (glaucoma + SOC) |
| 6 | IC TGFβ + Dextran Sulphate fluid gel (Fgel) ED |
| 6 | IC TGFβ + Fucoidan Fgel ED |
| 4 | IC TGFβ + Heparan Sulphate Fgel ED |

### Results of the In Vivo Glaucoma Study

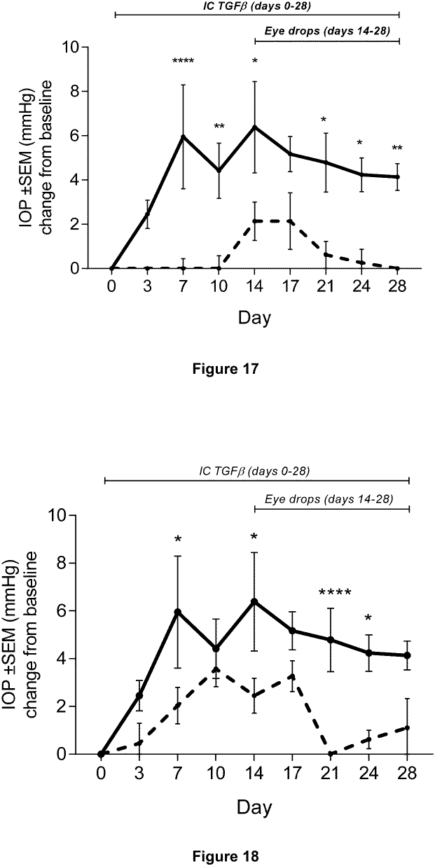

FIGS. 17 to 23 set out the results of a study investigating change in intraocular pressure (IOP) in the rodent model of glaucoma described above with one of the following:
SOC;
a dextran sulphate;
fucoidan; or
heparan sulphate treatments.

The composition comprising dextran sulphate, fucoidan and heparan sulphate are used as exemplary composition of the invention comprising a sulphated polysaccharide in a shear-thinning fluid gel.

FIG. 17 shows results achieved when administering SOC in glaucomatous eyes. SOC significantly reduced IOP at the end of the experiment at day 28 compared to glaucomatous eyes without any treatment.

FIG. 18 shows results achieved when experimental composition of the invention comprising the fluid gel containing dextran sulphate was administered as an eye drop. Here it can be seen that with passage of time, the IOP in the treated animals with glaucoma was significantly lower than in the glaucoma without any treatment.

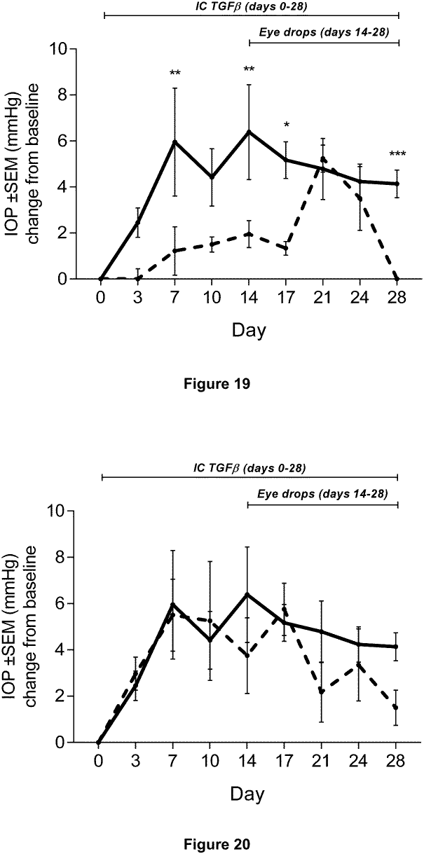

FIG. 19 shows results achieved when experimental composition of the invention comprising the fluid gel containing fucoidan was administered as an eye drop. Here it can be seen that with passage of time, the IOP in the treated animals with glaucoma was significantly lower than in the glaucoma without any treatment.

FIG. 20 shows results achieved when the experimental composition of the invention comprising fluid gel containing heparan sulphate was administered as an eye drop. Here it can be seen that with passage of time, the IOP in the treated animals with glaucoma was lower compared glaucoma without any treatment.

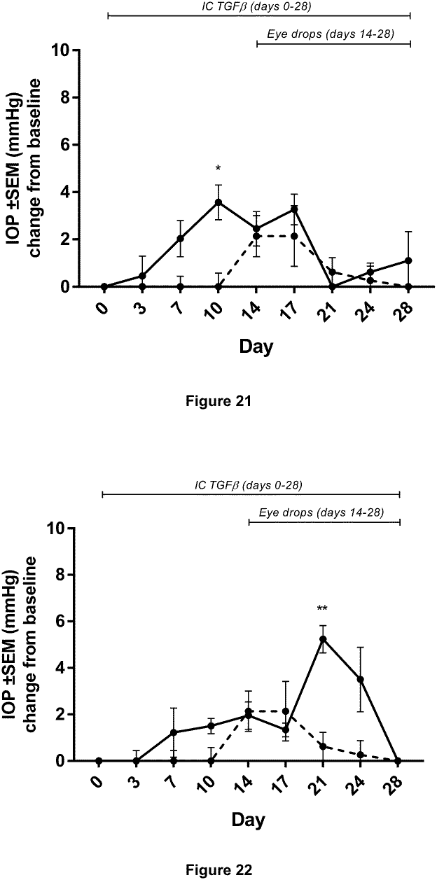

FIG. 21 shows results achieved when the experimental composition of the invention comprising fluid gel containing dextran sulphate was compared to the SOC. Here it can be seen that with passage of time, the IOP in animals treated with the fluid gel eye drop containing dextran sulphate was as effective to the standard of care by day 28.

FIG. 22 shows results achieved when the experimental composition of the invention comprising fluid gel containing fucoidan was compared to the SOC. Here it can be seen that with passage of time, the IOP in animals treated with the fluid gel eye drop containing fucoidan was as effective as the standard of care by day 28.

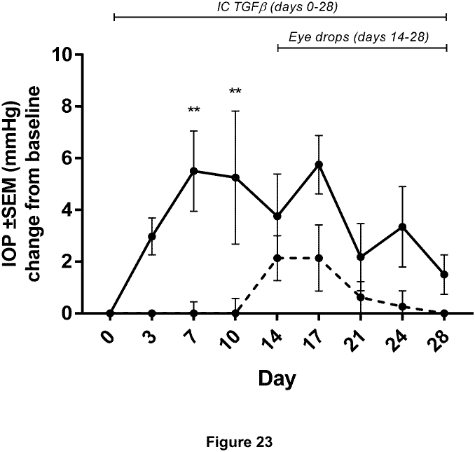

FIG. 23 shows results achieved when the experimental composition of the invention comprising fluid gel containing heparan sulphate was compared to the SOC. Here it can be seen that with passage of time, the IOP in animals treated with the fluid gel eye drop containing heparan sulphate was effective to the standard of care by day 28.

SOC treatment using latanoprost is helpful in addressing symptoms of glaucoma, by opening alternative drainage channels, thus increasing outflow of aqueous fluid from the eye in a manner that reduces IOP. However, it will be appreciated that this does not resolve the fibrosis that is the underlying cause of the increased IOP, and it is also known that patients’ response to SOC decreases with time. The compositions of the invention, by delivering sulphated polysaccharides such as dextran sulphate that can reduce fibrosis, not only provide relief equivalent to that provided by SOC in the short term (reduction in symptoms being equivalent within around 28 days), but also offer the prospect of long term resolution of the dysfunction of the eye’s main drainage pathways that causes elevated IOP, and hence offer a more complete and longer lasting therapeutic solution than the current SOC.

### Paragraphs Describing the Invention

The following paragraphs further describe the subject matter of the present invention, and may be taken into account when considering any of the statements of invention above.

1. A pharmaceutical composition comprising a sulphated polysaccharide and a shear-thinning fluid gel.

2. A composition according to paragraph 1 for use as a topical eyedrop in the treatment of glaucoma.

3. A composition according to paragraph 2 for use in the treatment of glaucoma by inhibition or reduction of fibrosis.

4. A composition according to paragraph 1 for use as a topical medicament in the inhibition or reduction of fibrosis.

5. A composition according to any preceding paragraph wherein the sulphated polysaccharide has an average sulphate number per repeating saccharide unit of between 1 and 3.

6. A composition according to any preceding paragraph wherein the sulphated polysaccharide is selected from the group consisting of: a dextran sulphate having an average molecular weight of 10,000 Da or less; heparan sulphate; fucoidan; poligeenan; furcellaran; and a carrageenan.

7. A composition according to paragraph 6 wherein the dextran sulphate has a number average molecular weight, Mn, as measured by nuclear magnetic resonance spectroscopy with an interval of 1850 and 3,500 D.

8. A composition according to paragraph 6 or paragraph 7 wherein the dextran sulphate has an average sulphate number per glucose unit of between 2.5 and 3.0.

9. A composition according to any of paragraphs 6 to 8 wherein the dextran sulphate has an average sulphation of C2 position in the glucose units of the dextran sulphate of at least 90%.

10. A composition according to any of paragraphs 1 to 6 wherein the sulphated polysaccharide has a molecular weight of between 5 and 1,000 kDa.

11. A composition according to paragraph 10 wherein the sulphated polysaccharide has a molecular weight of between 8 and 800 kDa.

12. A composition according to paragraph 6 wherein the carrageenan is selected from the group consisting of: iota carrageenan; kappa carrageenan; and lambda carrageenan.

13. A composition according to any preceding paragraph wherein the shear-thinning fluid gel has a viscosity of 0.1 Pa.s or greater when exposed to zero shear and the viscosity reduces when the shear-thinning fluid gel is subjected to shear.

14. A composition according to paragraph 13 wherein the shear-thinning fluid gel has a viscosity of 5 Pa.s or greater when exposed to zero shear and the viscosity reduces to below 1 Pa.s when the shear-thinning fluid gel is subjected to shear.

15. A composition according to any preceding paragraph wherein the shear-thinning fluid gel comprises a shear-thinning hydrogel composition comprising 0.1 to 10% w/v (such as 0.1 to 5% w/v, or 0.1 to 2.5% w/v) of a microgel particle forming polymer dispersed in an aqueous medium, wherein the viscosity and/or elastic modulus of the shear-thinning composition reduces when the hydrogel is exposed to shear.

16. A composition according to paragraph 15 wherein the shear-thinning hydrogel composition further comprises 0.5 to 100 mM of a monovalent and/or polyvalent metal ion salt as a cross-linking agent.

17. A composition according to any preceding paragraph wherein the shear-thinning fluid gel comprises a microgel particle forming polymer selected from one or more of the following groups: gellans; alginates; carrageenans; agarose; chitosan; pectin; agarose; agar or gelatin.

18. A composition according to any preceding paragraph wherein the shear-thinning fluid gel comprises gellan.

19. A composition according to paragraph 18 wherein the shear-thinning fluid gel comprises approximately 0.9% w/v of gellan.

20. A composition according to any of paragraphs 1 to 16 wherein the shear-thinning fluid gel comprises a synthetic microgel particle forming polymer selected from one or more of: polyols (such as polyalkylene glycols); polyamides; polyesters; polyalkylenes; polystyrenes and polyacrylates.

21. A sulphated polysaccharide for use in combination with a shear-thinning fluid gel in the prevention and/or treatment of glaucoma.

22. A sulphated polysaccharide for use in combination with a shear-thinning fluid gel in the inhibition or reduction of fibrosis.

23. A sulphated polysaccharide for use according to paragraph 21 or 22 wherein the sulphated polysaccharide is selected from the group consisting of: a dextran sulphate having an average molecular weight of 10,000 Da or less; heparan sulphate; poligeenan; furcellaran; and a carrageenan.

24. A sulphated polysaccharide for use according to any of paragraphs 21 to 23 wherein the shear-thinning fluid gel is as defined in any of paragraphs 13 to 20.

25. A sulphated polysaccharide for use according to any of paragraphs 21 to 24 wherein the shear-thinning fluid gel comprises gellan.

1. A pharmaceutical composition comprising a sulphated polysaccharide and a shear-thinning fluid gel.

2. A composition according to claim 1 for use in the prevention and/or treatment of glaucoma.

3. A composition according to claim 2 for use as a topical medicament in the treatment of glaucoma.

4. A composition according to claim 2 or claim 3 for use as a topical eyedrop in the treatment of glaucoma.

5. A composition according to any of claims 2 to 4 for use in the treatment of glaucoma by inhibition or reduction of fibrosis.

6. A composition according to claim 1 for use in the inhibition or reduction of fibrosis.

7. A composition according to claim 6 for use as a topical medicament in the inhibition or reduction of fibrosis.

8. A composition according to any of claims 1 to 7 wherein the sulphated polysaccharide has an average sulphate number per repeating saccharide unit of between 1 and 3.

9. A composition according to any of claims 1 to 8 wherein the sulphated polysaccharide is selected from the group consisting of: a dextran sulphate having an average molecular weight of 10,000 Da or less; heparan sulphate; fucoidan; poligeenan; furcellaran; and a carrageenan.

10. A composition according to claim 9 wherein the dextran sulphate has a number average molecular weight, Mn, as measured by nuclear magnetic resonance spectroscopy with an interval of 1850 and 3,500 D.

11. A composition according to claim 9 or claim 10 wherein the dextran sulphate has an average sulphate number per glucose unit of between 2.5 and 3.0.

12. A composition according to any of claims 9 to 11 wherein the dextran sulphate has an average sulphation of C2 position in the glucose units of the dextran sulphate of at least 90%.

13. A composition according to any of claims 1 to 9 wherein the sulphated polysaccharide has a molecular weight of between 5 and 1,000 kDa.

14. A composition according to claim 13 wherein the sulphated polysaccharide has a molecular weight of between 8 and 800 kDa.

15. A composition according to claim 13 or 14 wherein the sulphated polysaccharide comprises heparan sulphate.

16. A composition according to claim 15 wherein the heparan sulphate has a molecular weight of approximately 8 kDa.

17. A composition according to claim 15 or 16 wherein the heparan sulphate has an average sulphate number per disaccharide unit of between 2.4 and 2.7.

18. A composition according to claim 13 or 14 wherein the sulphated polysaccharide comprises fucoidan.

19. A composition according to claim 18 wherein the fucoidan has a molecular weight of approximately 20 kDa.

20. A composition according to claim 18 or 19 wherein the fucoidan has an average sulphate number per disaccharide unit of between 1 and 2.

21. A composition according to claim 13 or 14 wherein the sulphated polysaccharide comprises between 15% and 40% estersulphate by weight.

22. A composition according to claim 21 wherein the sulphated polysaccharide comprises between 20% and 35% estersulphate by weight.

23. A composition according to claim 13, 14, 21 or 22 wherein the sulphated polysaccharide is selected from the group consisting of: poligeenan; furcellaran; and a carrageenan.

24. A composition according to claim 23 wherein the sulphated polysaccharide comprises poligeenan.

25. A composition according to claim 24 wherein the poligeenan has a molecular weight of between 20 and 30 kDa.

26. A composition according to claim 23 wherein the sulphated polysaccharide comprises furcellaran.

27. A composition according to claim 26 wherein the furcellaran has a molecular weight of between 20 and 80 kDa.

28. A composition according to claim 23 wherein the sulphated polysaccharide comprises a carrageenan.

29. A composition according to claim 28 wherein the carrageenan is selected from the group consisting of: iota carrageenan; kappa carrageenan; and lambda carrageenan.

30. A composition according to claim 29 wherein the carrageenan comprises iota carrageenan.

31. A composition according to claim 30 wherein the average sulphate number per disaccharide unit of the iota carrageenan is 2.

32. A composition according to claim 29 wherein the carrageenan comprises kappa carrageenan.

33. A composition according to claim 32 wherein the average sulphate number per disaccharide unit of the kappa carrageenan is 1.

34. A composition according to claim 29 wherein the carrageenan comprises lambda carrageenan.

35. A composition according to claim 34 wherein the average sulphate number per disaccharide unit of the lambda carrageenan is 3.

36. A composition according to any preceding claim wherein the shear-thinning fluid gel has a viscosity of 0.1 Pa.s or greater when exposed to zero shear and the viscosity reduces when the shear-thinning fluid gel is subjected to shear.

37. A composition according to claim 36 wherein the shear-thinning fluid gel has a viscosity of 5 Pa.s or greater when exposed to zero shear and the viscosity reduces to below 1 Pa.s when the shear-thinning fluid gel is subjected to shear.

38. A composition according to any preceding claim wherein the shear-thinning fluid gel comprises a shear-thinning hydrogel composition comprising 0.1 to 10% w/v (such as 0.1 to 5% w/v, or 0.1 to 2.5% w/v) of a microgel particle forming polymer dispersed in an aqueous medium, wherein the viscosity and/or elastic modulus of the shear-thinning composition reduces when the hydrogel is exposed to shear.

39. A composition according to claim 38 wherein the shear-thinning hydrogel composition further comprises 0.5 to 100 mM of a monovalent and/or polyvalent metal ion salt as a crosslinking agent.

40. A composition according to any preceding claim wherein the shear-thinning fluid gel comprises a microgel particle forming polymer selected from one or more of the following groups: gellans; alginates; carrageenans; agarose; chitosan; pectin; agarose; agar or gelatin.

41. A composition according to any preceding claim wherein the shear-thinning fluid gel comprises gellan.

42. A composition according to claim 41 wherein the shear-thinning fluid gel comprises approximately 0.9% w/v of gellan.

43. A composition according to any of claims 1 to 39 wherein the shear-thinning fluid gel comprises a synthetic microgel particle forming polymer selected from one or more of: polyols (such as polyalkylene glycols); polyamides; polyesters; polyalkylenes; polystyrenes and polyacrylates.

44. A composition according to any of claims 1 to 43 wherein the shear-thinning fluid gel is a non-sulphated fluid gel.

45. A composition according to any of claims 1 to 44 wherein the microgel particle forming polymer of the shear-thinning fluid gel is a non-sulphated polymer.

46. A sulphated polysaccharide for use in combination with a shear-thinning fluid gel in the prevention and/or treatment of glaucoma.

47. A sulphated polysaccharide for use in combination with a shear-thinning fluid gel in the inhibition or reduction of fibrosis.

48. A sulphated polysaccharide for use according to claim 46 or 47 wherein the sulphated polysaccharide is as defined in any of claims 8 to 35.

49. A sulphated polysaccharide for use according to any of claims 46 to 48 wherein the sulphated polysaccharide is selected from the group consisting of: a dextran sulphate having an average molecular weight of 10,000 Da or less; heparan sulphate; poligeenan; furcellaran; and a carrageenan.

50. A sulphated polysaccharide for use according to any of claims 46 to 49 wherein the shear-thinning fluid gel is as defined in any of claims 36 to 45.

51. A sulphated polysaccharide for use according to any of claims 46 to 50 wherein the shear-thinning fluid gel comprises gellan.

52. A shear-thinning fluid gel for use with a sulphated polysaccharide in the prevention and/or treatment of glaucoma.

53. A shear-thinning fluid gel for use with a sulphated polysaccharide in the inhibition or reduction of fibrosis.

54. A shear-thinning fluid gel for use according to claim 50 or 51 wherein the shear-thinning fluid gel is as defined in any of claims 36 to 45.

55. A shear-thinning fluid gel for use according to any of claims 52 to 54 wherein the shear-thinning fluid gel comprises gellan.

56. A shear-thinning fluid gel for use according to any of claims 52 to 55 wherein the sulphated polysaccharide is as defined in any of claims 8 to 35.

57. A shear-thinning fluid gel for use according to any of claims 52 to 56 wherein the sulphated polysaccharide is selected from the group consisting of: a dextran sulphate having an average molecular weight of 10,000 Da or less; heparan sulphate; poligeenan; furcellaran; and a carrageenan.

58. A sulphated polysaccharide selected from the group consisting of: heparan sulphate; poligeenan; furcellaran; and a carrageenan, for use in the inhibition or reduction of fibrosis.

59. A sulphated polysaccharide comprising heparan sulphate for use in the inhibition or reduction of fibrosis.

60. A sulphated polysaccharide comprising a carrageenan, for use in the inhibition or reduction of fibrosis.

61. A sulphated polysaccharide for use according to any of claims 58 to 60 as a topical medicament.

62. A sulphated polysaccharide for use according to any of claims 58 to 61 as a topical eyedrop.

63. A gellan shear-thinning fluid gel for use as an eyedrop in the prevention and/or treatment of glaucoma.

64. A gellan shear-thinning fluid gel for use in the inhibition or reduction of fibrosis.

65. A gellan shear-thinning fluid gel for use according to claim 63 or claim 64 wherein the shear-thinning fluid gel comprises approximately 0.9% w/v of gellan.
